(19) United States
(12) Patent Application Publication
(10) Pub. No.: US 2005/0160511 A1
(43) Pub. Date: Jul. 28, 2005

(54) Sunglasses attachable to cap

(76) Inventor: Kyung Nam Kim, Susung-Gu (KR)

Correspondence Address: PIPER RUDNICK LLP, P. O. BOX 9271, RESTON, VA, 20195, US

(21) Appl. No.: 10/975,847

(22) Filed: Oct. 29, 2004

(30) Foreign Application Priority Data
Jan. 28, 2004 (KR) 20-2004-0002010

(51) Int. Cl. A61F 9/00

(52) U.S. Cl. 2/10

(57) ABSTRACT

Disclosed herein are sunglasses attachable to a cap, which are more readily adjustable corresponding to the size of a cap visor. The sunglasses comprises a frame with an integral lens unit suspending downwardly from a lower side thereof, and two clamps provided at the frame and adapted to grasp a cap visor. Each of the clamps includes a support extending vertically from one of both side positions equally spaced apart from the center of an upper surface of the frame, a joint extending outwardly from the support and having a ball formed at a distal end thereof, and a clamping body located outward of the joint. The clamping body has a visor clamping pin, which defines a visor groove for securely grasping either side brim of the cap visor, and a ball socket defined at an inner surface.

### BACKGROUND OF THE INVENTION

1. Field of the Invention

The present invention relates to sunglasses attachable to a cap, and more particularly to sunglasses for a cap, which can be easily attached to any of kind cap.

2. Description of the Related Art

Recently, sunglasses, which have been increasingly used, are generally used to intercept sunlight in the summer having high intensity of direct light and ultraviolet light.

Such sunglasses are classified into ordinary sunglasses with tinted lenses, which are for persons not wearing prescription lenses, and sunshade clips wherein a tinted lens unit of the ordinary sunglasses is detachably attached to ordinary prescription lenses, which are only for persons having to wear the prescription lenses.

In addition to the above-described types, currently, various kinds of sunglasses, for example, sunglasses attachable to a cap, have been developed and used. The sunglasses attachable to a cap are configured to allow a wearer to attach the sunglasses to a forward projecting visor or bill of the cap after wearing the cap.

Considering a general configuration of the conventional sunglasses attachable to a cap with reference to FIGS. 1 and 2, the sunglasses, which are designated as reference numeral 3, comprise a frame 2 with an integral lens unit 1 suspending downwardly from a lower end thereof, and two clamps 5 integrally mounted to the frame 2 and adapted to grasp the cap visor 4.

The clamps 5 have supports 6 extending vertically from both side positions equally spaced apart from the center of an upper surface of the frame 2, respectively, and joints 8 extending in opposite outward directions from the supports 6, respectively. The joints 8 have balls 7 formed at their distal ends, respectively.

Each of the clamps 5 also has a clamping body 12 positioned outward of an associated one of the joints 8. The clamping body 12, in turn, has a visor clamping pin 11, which is configured to define a visor groove 10 for securely grasping either side brim of the visor 4 provided at a cap 9. At an inner surface of the clamping body 12 is defined a ball socket 13 for the interference fit of the ball 7 of the joint 8 while securing free movement of the ball 7 therein.

With the above described prior art, however, since a distance between both the clamps 5 mounted at the sides of the frame 2 is unchangeable, it is difficult for the conventional sunglasses to conform to different sized cap visors.

Therefore, there is a need to manufacture various sizes of sunglasses to conform to any size adults caps and children's caps, and this becomes a factor of unnecessarily increasing not only costs of products but also user's inconvenience.

### SUMMARY OF THE INVENTION

Therefore, the present invention has been made in view of the above problems, and it is an object of the present invention to provide sunglasses attachable to a cap, which are more readily adjustable corresponding to the size of a cap visor, thereby enabling the sunglasses to be commonly used regardless of whether the caps are adults caps or children's caps, resulting in not only reduction of manufacturing cost but also increase of convenience for users.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a front view illustrating sunglasses attachable to a cap in accordance with the prior art;

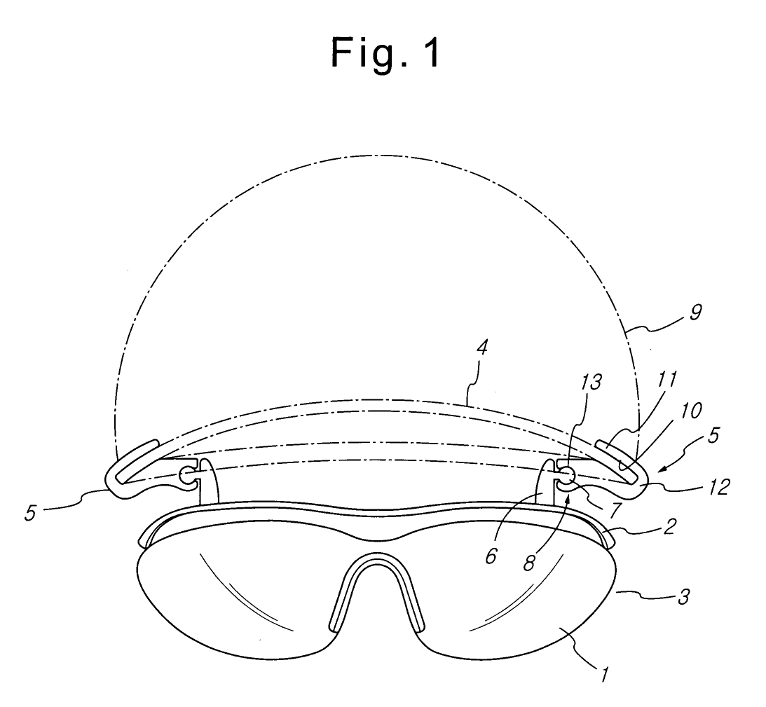

FIG. 2 is an enlarged front sectional view illustrating a clamp as an important constituent of the sunglasses attachable to a cap shown in FIG. 1;

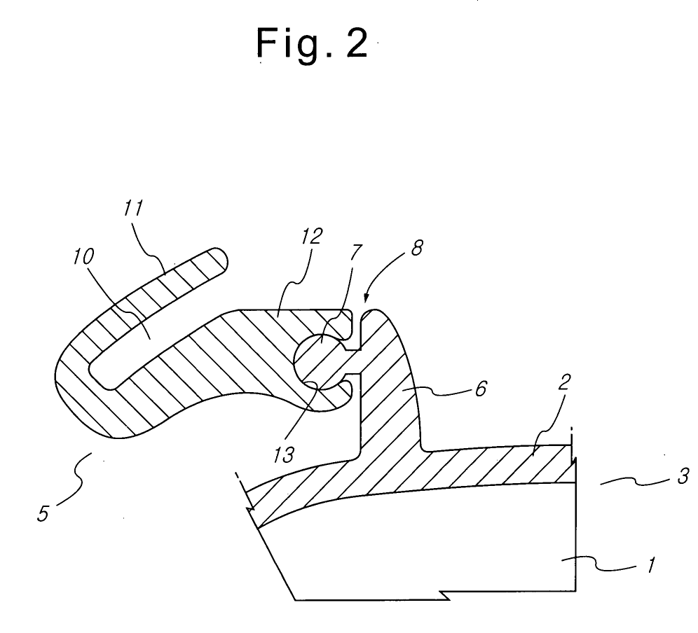

FIG. 3 is a perspective view illustrating sunglasses attachable to a cap in accordance with the present invention;

FIG. 4 is an exploded perspective view illustrating a clamp as an important constituent of the sunglasses attachable to a cap in accordance with the present invention;

FIG. 5 is a sectional view illustrating the clamp of the sunglasses attachable to a cap in accordance with the present invention; and

FIG. 6 is a perspective view illustrating the sunglasses, in accordance with the present invention, attached to a cap for use thereof.

### DESCRIPTION OF THE PREFERRED EMBODIMENTS

Now, a preferred embodiment of the present invention will be described in detail with reference to the annexed drawings.

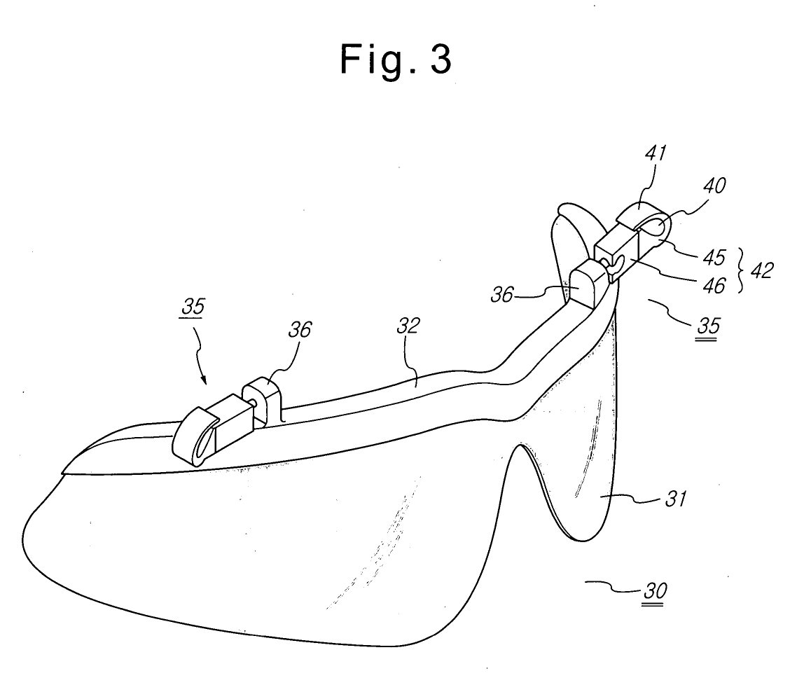

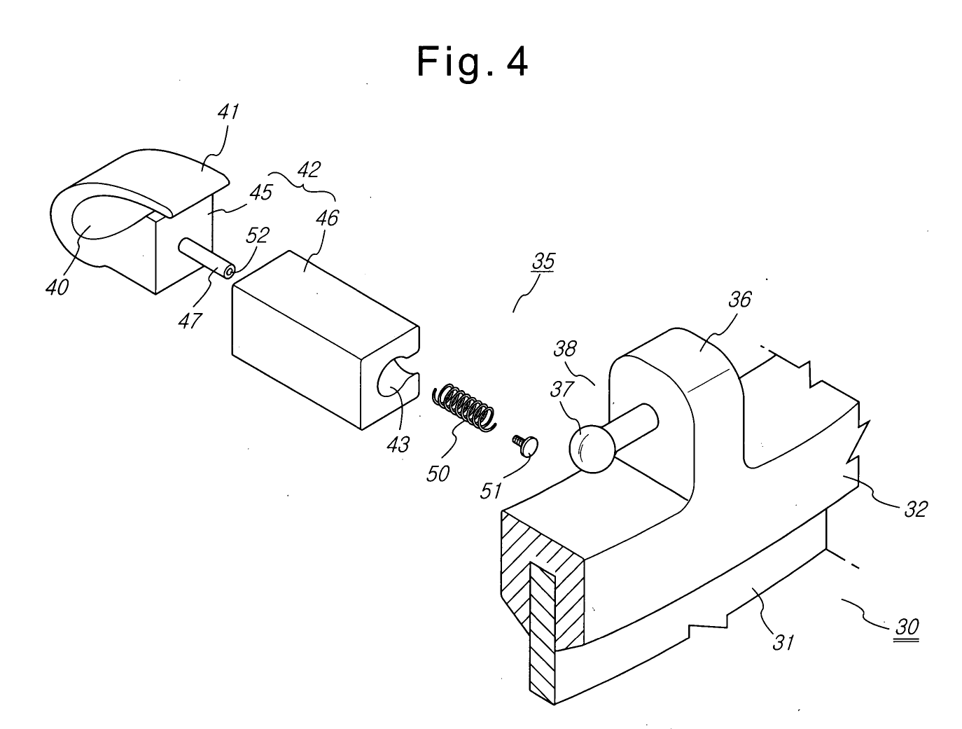

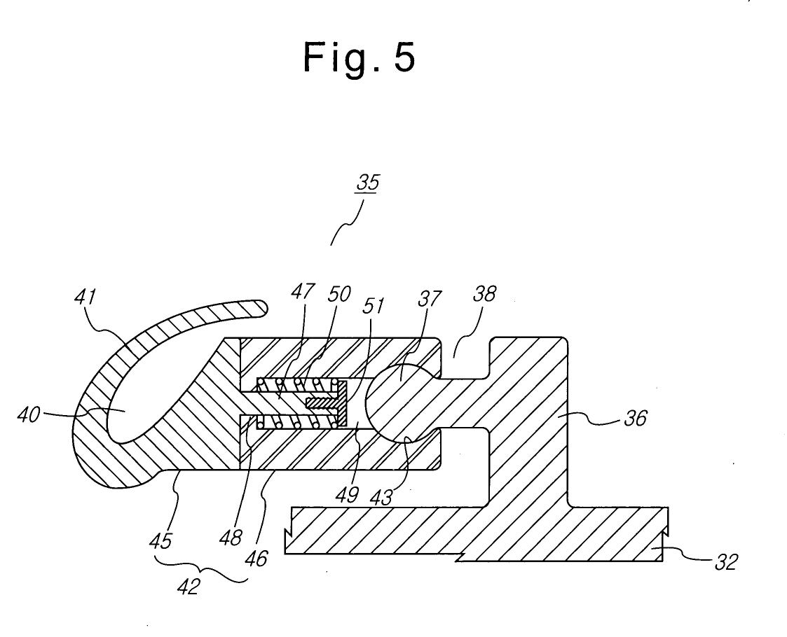

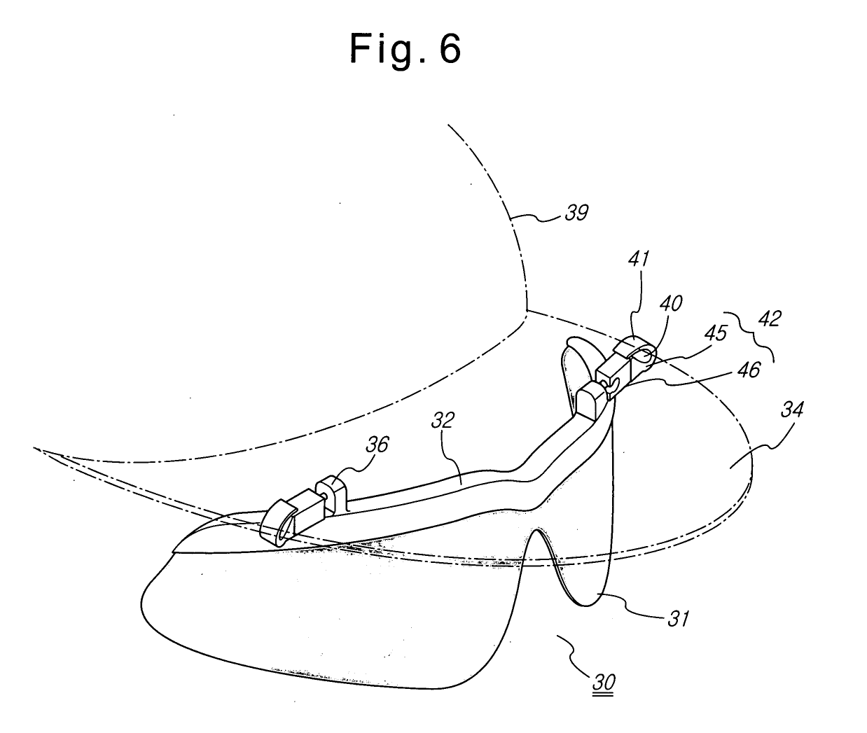

FIG. 3 is a perspective view illustrating sunglasses attachable to a cap in accordance with the present invention. FIG. 4 is an exploded perspective view illustrating a clamp as an important constituent of the sunglasses attachable to a cap in accordance with the present invention. FIG. 5 is a sectional view illustrating the clamp of the sunglasses attachable to a cap in accordance with the present invention. FIG. 6 is a perspective view illustrating the sunglasses, in accordance with the present invention, attached to a cap for use thereof. First, the configuration of the present invention will be explained.

The sunglasses attachable to a cap in accordance with the present invention, which are designated as reference numeral 30, comprise a frame 32 with an integral lens unit 31 suspending downwardly from a lower end thereof, and two clamps 35 integrally mounted to the frame 32 and adapted to grasp a cap visor 34. The two clamps 35 are the same, one of which will accordingly be described hereinafter.

Each of the clamps 35 has a support 36 extending vertically from one of both side positions equally spaced apart from the center of an upper surface of the frame 32, and a joint 38 extending outwardly from the support 36. The joint 38 has a ball 37 formed at a distal end thereof.

The clamp 35 also has a clamping body 42 positioned outward of the joint 38. The clamping body 42 has a visor clamping pin 41, which is configured to define a visor groove 40 for securely grasping either side brim of the visor 34 provided at a cap 39. At an inner surface of the clamping body 42 is defined a ball socket 43 for the interference fit of the ball 37 of the joint 38 while securing free movement of the ball 37 therein.

In the present embodiment, the clamp 35 has an important improved feature in that it can be more readily and positively conformed to different sizes of the visor 34 provided at the cap 39.

In order to achieve such a feature of the clamp 35, according to the present invention, the clamping body 42 of the clamp 35 is divided into two components, for example, an outer body 45 formed with the visor clamping pin 41, and an inner body 46 formed with the ball socket 43. The outer body 45 is further formed with an actuating bar 47 at an inner surface thereof facing the inner body 46. The actuating bar 47 protrudes from the center of the inner surface of the outer body 45 toward the inner body 46.

In order to allow the actuating bar 47 to be inserted therein, the inner body 46 has an actuating groove 49, which is externally defined to penetrate throughout the inner body 46. The actuating groove 49 extends from the ball socket 43 defined at one distal end of the inner body 46 to a stepped holding portion 48 defined at the other distal end thereof. The actuating groove 49 has a diameter smaller than a diameter of the ball socket 43.

Around the actuating bar 47 is fitted a spring 50, and then a fastener 51 is fastened into a screw bore 52 internally defined in an inner end region of the actuating bar 47. Under such an arrangement, the outer body 45 is displaceable by virtue of an elastic force of the spring 50.

Now, the operation of the sunglasses attachable to a cap in accordance with the present invention will be explained.

In an initial state, the clamp 35 of the sunglasses 30 attachable to a cap, causes the outer body 45 and the inner body 46 to come into close contact with each other by means of the elastic force of the spring 50.

Then, as the visor groove 40 of the visor clamping pin 41 formed at the outer body 45 grasps either side brim of the cap visor 34, the sunglasses 30 are completely attached to the cap 39.

In such an attached state, the sunglasses 30 are maintained in an operative position suspending downward from the visor 34 so as to intercept sunlight, or are pivoted upward into a non-operative or storage position horizontally residing adjacent to the underside of the visor 34.

Such a pivotal movement of the sunglasses 30 is enabled since the ball 37 of the joint 38 is pivotably interference fitted in the ball socket 43 of the inner body 46.

Especially, by means of the spring 50, which is fitted around the actuating bar 47 located inside the actuating groove 49 of the inner body 46 by using the fastener 51, when the sunglasses 30 are attached to the visor 34 of the cap 39, the outer body 45 is easily extendible or retractable. This facilitates the attachment of the sunglasses 30.

Furthermore, this enables not only the sunglasses 30 to be easily conformed to a variation in the size of the visor 34 of the cap 39 within an extendible/retractable range of the outer body 45, but also the sunglasses 30 attached to the cap 39 to continuously maintain the firm attached state by means of a restoring force of the spring 50.

The above described sunglasses attachable to a cap according to the present invention can be commonly usable in any kinds of caps having a visor or bill regardless of their sizes. Therefore, the present invention achieves outstanding advantages in view of productivity and manufacturing cost, and is easy in relation to attachment to a cap.

As apparent from the above description, the present invention provides sunglasses attachable to a cap, which are more readily adjustable corresponding to the size of a cap visor, thereby enabling the sunglasses to be commonly used regardless of whether the caps are adults caps or children's caps, resulting in not only reduction of manufacturing cost but also increase of convenience for users.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. Sunglasses attachable to a cap comprising: a frame with an integral lens unit suspending downwardly from a lower side thereof; and two clamps provided at the frame and adapted to grasp a cap visor,
wherein each of the clamps includes:
a support extending vertically from one of both side positions equally spaced apart from the center of an upper surface of the frame;
a joint extending outwardly from the support and having a ball formed at a distal end thereof; and
a clamping body located outward of the joint, the clamping body having a visor clamping pin, which defines a visor groove for securely grasping either side brim of the cap visor, and a ball socket defined at an inner surface of the clamping body facing the ball for the interference fit of the ball while securing pivotal movement of the ball therein,
wherein the clamping body includes:
an outer body formed with the visor clamping pin, and an actuating bar protruding from an inner surface thereof toward the inner body; and
an inner body formed with the ball socket, and an actuating groove for the insertion of the actuating bar, the actuating groove extending from the ball socket of the inner body and having a diameter smaller than a diameter of the ball socket, a stepped holding potion being defined at an end of the actuating groove, and,
wherein the outer body is displaceable to conform to the size of the cap by means of a spring, which is fitted around the actuating bar, and is clamped by a fastener.
